(19) United States
(12) Patent Application Publication
(10) Pub. No.: US 2015/0328215 A1
(43) Pub. Date: Nov. 19, 2015

(54) STABLE AMORPHOUS RALTEGRAVIR POTASSIUM PREMIX AND PROCESS FOR THE PREPARATION THEREOF

(71) Applicant: LUPIN LIMITED, Mumbai, Maharashtra (IN)

(72) Inventors: Surinder Kumar ARORA, Pune, Maharashtra (IN); Dnyaneshwar Tukaram SINGARE, Pune, Maharashtra (IN); Amol Dattatreya GALANDE, Pune, Maharashtra (IN); Purna Chandra RAY, Pune, Maharashtra (IN); Dilip Gopalkrishna SAOJI, Pune, Maharashtra (IN); Makrand AVACHAT, Pune, Maharashtra (IN); Girij Pal SINGH, Pune, Maharashtra (IN)

(21) Appl. No.: 14/373,940

(22) PCT Filed: Jan. 25, 2013
(86) PCT No.: PCT/IB2013/050643

§ 371 Date: Jul. 23, 2014

(30) Foreign Application Priority Data
Jan. 25, 2012 (IN) 72/KOL/2012

(51) Int. Cl. A61K 31/513; A61K 45/06; A61K 9/14

(52) U.S. Cl. CPC A61K 31/513; A61K 9/145; A61K 9/146; A61K 9/143; A61K 45/06

(57) ABSTRACT

The present invention relates to a stable amorphous Raltegravir potassium premix, method of making and pharmaceutical composition thereof.

### TECHNICAL FIELD OF INVENTION

The present invention is directed to a stable amorphous Raltegravir potassium premix, method of making and pharmaceutical composition thereof.

### BACKGROUND OF THE INVENTION

Raltegravir potassium is chemically known as potassium N-(4-fluorobenzyl)-5-hydroxy-1-methyl-2-(1-methyl-1-{[(5-methyl-1,3,4-oxadiazol-2-yl)carbonyl]amino}ethyl)-6-oxo-1,6-dihydropyrimidine-4-carboxamide. Raltegravir potassium is a potent HL1 integrase inhibitor which is used for treatment of HL1 infections, AIDS, and Aids Related Complex (ARC).

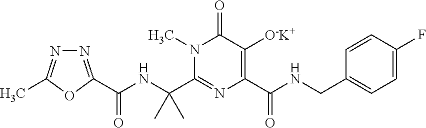

Raltegravir is generically disclosed in U.S. Pat. No. 7,169,780 B2 and potassium salt of Raltegravir is specifically described by U.S. Pat. No. 7,754,731 B2. Raltegravir exhibits poor aqueous solubility where as the potassium salt of Raltegravir is significantly more soluble in water and exhibit improved pharmacokinetics in animal models over Raltegravir free base.

Polymorphism is the ability of a compound to exist in two or more different crystalline phasesthat differ in arrangement of the molecules in crystal lattice. Although polymorphs have the same chemical composition, they differ in packing and geometrical arrangement and exhibit different physical properties such as melting point, X-ray diffraction patterns, density, stability, and solubility.

Extensive study is carried out in pharmaceutical industry for development of different polymorphs of various drug substances, to obtain suitable polymorphs that possess improved performance characteristics such as aqueous solubility, improved bioavailability, chemical stability, shelf life etc.

Literature survey reveals that Raltegravir potassium can exist in different polymorphic forms, which differ from each other in terms of stability, physical properties and pharmacokinetics. Very few documents in prior art are directed towards polymorphs of Raltegravir potassium, which are incorporated here by way of reference.

The PCT application WO 2006/060712 A2 discloses two anhydrous crystalline forms of Raltegravir potassium viz., form 1 and form 3 and one crystalline hydrate designated as form 2. Form 1 is especially known to exhibit superior bioavailability and improved pharmacokinetics over Raltegravir free base. It can be prepared by crystallization of Raltegravir potassium from a mixture of potassium base, Raltegravir, water and an alcohol.

Hydrated crystalline form 2 is prepared by sonicating a mixture of Raltegravir, KOH, acetone and trace amount of water whereas anhydrous crystalline form 3 is obtained by crystallization of amorphous Raltegravir potassium from ethanol.

The PCT application WO 2010/140156 A2 describes amorphous form and crystalline form H1 of Raltegravir potassium. The process for preparation of crystalline form H1 comprises of providing a solution of Raltegravir potassium in dimethyl formamide, dimethyl acetamide or mixtures thereof and further separating and isolating the solid obtained. The amorphous form is obtained by freeze drying the aqueous solution of Raltegravir potassium at −180° C. Although the description mentions preparation of amorphous Raltegravir potassium by spray drying method it does not provide an enabling disclosure.

Though amorphous Raltegravir potassium and its process of manufacture has been described in Patent Application WO 2010/140156 A2, Raltegravir potassium in premix form, is a novel approach by the present inventors towards attaining a significantly more stable amorphous product having better dissolution properties that can be easily formulated to give pharmaceutical compositions.

Premixes are characterized by a variety of associated properties such as stability, flow, and solubility. Typical premixes represent a compromise of the above properties, as for example, an increase in stability and dissolution properties of the premix. Although there are a variety of premixes, there is a continual search in this field of art for premixes that exhibit an improved mix of properties. Thus, the instant invention provides a premix in which Raltegravir potassium exists in stable amorphous form and process of manufacture of the premix and pharmaceutical compositions comprising said Raltegravir potassium premix.

### DESCRIPTION OF DRAWINGS

FIG. 1: illustrates X-ray powder diffraction pattern of amorphous Raltegravir potassium premix with mannitol.

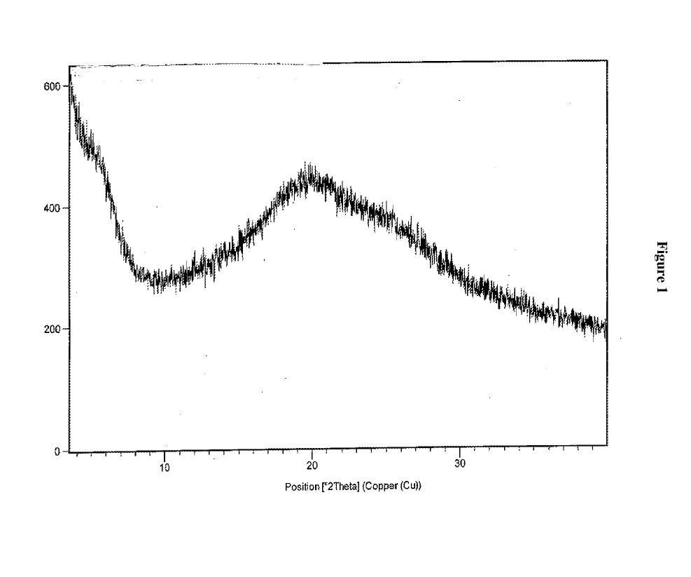

FIG. 2: illustrates X-ray powder diffraction pattern of amorphous Raltegravir potassium premix with PEG-4000

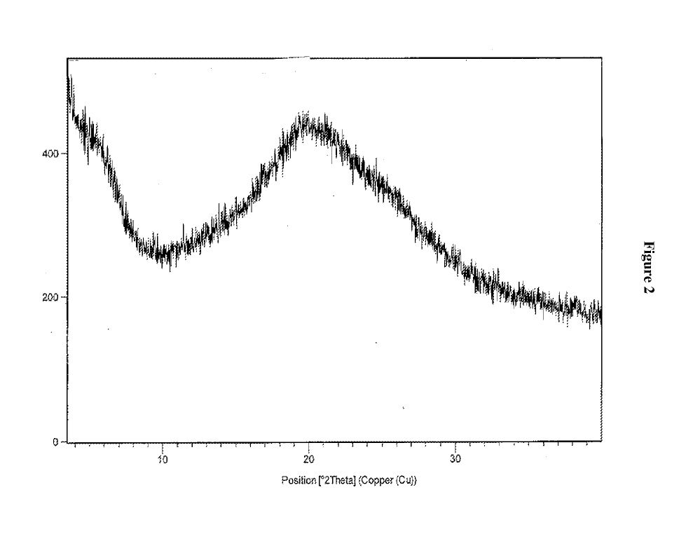

FIG. 3: illustrates X-ray powder diffraction pattern of amorphous Raltegravir potassium premix with Aerosil 200

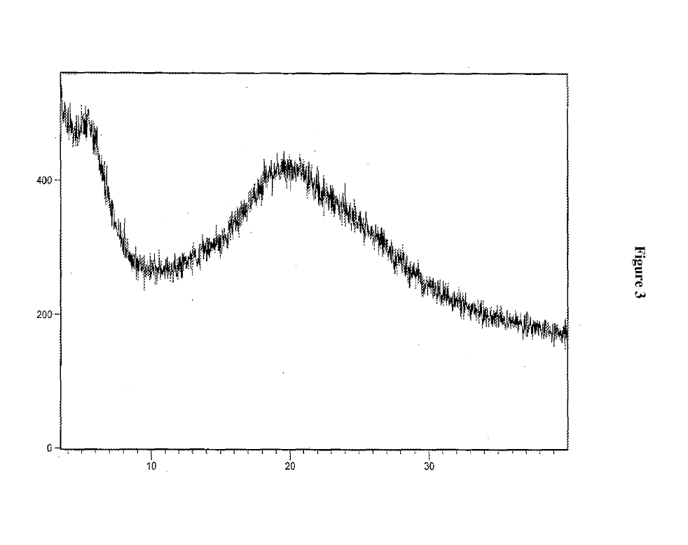

FIG. 4: illustrates X-ray powder diffraction pattern of amorphous Raltegravir potassium

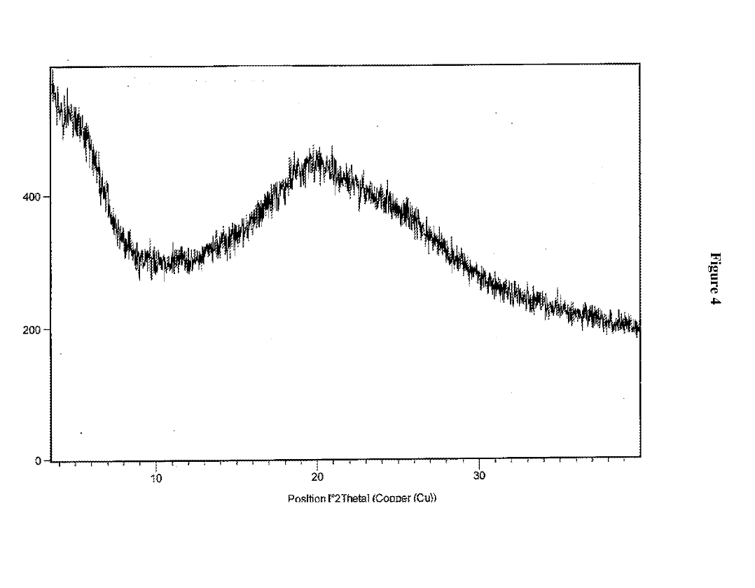

### SUMMARY OF THE INVENTION

In one aspect, the present invention provides stable amorphous Raltegravir potassium premix having enhanced stability and dissolution properties and process for preparation thereof.

In another aspect, the invention provides for pharmaceutical compositions comprising said stable amorphous Raltegravir potassium premix.

### DESCRIPTION OF THE INVENTION

The term “premix” is used herein to describe combinations of Raltegravir potassium and at least one pharmaceutically acceptable excipient, wherein individual particles of the components cannot be distinguished using techniques such as optical microscopy. In embodiments, the drug is considered as being uniformly or non-uniformly distributed over surfaces of excipient particles. In other embodiments, the premixes are considered to be in the nature of molecular dispersions, or solid solutions. Simple mixtures of powdered ingredients will not constitute premixes.

The term “excipient” or “pharmaceutically acceptable excipient” means a component of a pharmaceutical product that is not an active ingredient, and includes but not limited to filler, diluent, disintegrants, glidants, stabilizers, surface active agents etc. The excipients that are useful in preparing a pharmaceutical composition are generally safe, non-toxic and neither biologically nor otherwise undesirable, and are acceptable for veterinary use as well as human pharmaceutical use. One excipient can perform more than one function.

In one embodiment of the invention, the present invention provides a stable amorphous Raltegravir potassium premix having enhanced stability, dissolution properties that can be easily formulated into pharmaceutical compositions.

In another embodiment of the invention, the present invention provides a stable amorphous Raltegravir potassium premix comprising Raltegravir potassium and at least one pharmaceutically acceptable excipient.

Any of the pharmaceutically acceptable excipient described in the specification can be used in the process of preparing stable amorphous Raltegravir potassium premix.

The pharmaceutically acceptable excipients used in the process of preparing stable amorphous Raltegravir potassium premix may also be termed as “premixing agents”.

The stable amorphous premix can further be mixed with other pharmaceutically acceptable excipients to prepare a pharmaceutical formulation or composition of the present invention.

The suitable premixing agent or pharmaceutically acceptable excipient(s) discussed in the specification includes but not limited to diluents, lubricants, disintegrants, glidants, stabilizers & surface active agents or mixtures thereof. Preferably the premixing agents or pharmaceutically acceptable excipients used in the process of preparing stable amorphous Raltegravir potassium premix can be selected from the group consisting of polyvinylpyrrolidone (also called povidone), polyvinyl alcohol, polyethylene glycol, polyol(Mannitol), sodium starch glycolate, colloidal silicon dioxide(aerosil), hydroxypropyl methylcellulose, methyl cellulose, carboxymethyl cellulose, sodium carboxymethyl cellulose, hydroxyethylcellulose, polyvinyl acetate, cyclodextrins, gelatins, hypromellose phthalate, sugars and combinations comprising one or more of the foregoing agents, Preferably selected from povidone, mannitol, polyethylene glycol (PEG) and aerosol 200.

In another embodiment, the invention provides a process for preparation of stable amorphous Raltegravir potassium premix comprising the steps of:
(i) providing a solution of crystalline Raltegravir potassium in a solvent;
(ii) adding suitable premixing agent(s); and
(iii) substantially removing the solvents from the solution to afford stable amorphous Raltegravir potassium premix.

The term “substantially removing” the solvent refers to at least 80%, specifically greater than about 85%, more specifically greater than about 90%, still more specifically greater than about 99%, and most specifically essentially complete (100%), removal of the solvent from the solvent solution.

The solvent employed in step (i) is selected from halogenated hydrocarbons such as dichloromethane, 1,2-dichloroethane, chloroform and carbon tetrachloride; alcohols such as methanol, ethanol, isopropyl alcohol, 1-propanol, 2-propanol, 1-butanol, 2-butanol, and t-butyl alcohol; ketones such as acetone, ethyl methyl ketone, diethyl ketone, and methyl isobutyl ketone; esters such as ethyl acetate, n-propyl acetate, n-butyl acetate and t-butyl acetate; ethers such as diethyl ether, dimethyl ether, diisopropyl ether, methyl t-butyl ether and 1,4-dioxane; nitriles such as acetonitrile and propionitrile; water; and mixtures thereof;

The reaction of step (i) is carried out at a temperature of 0 to 50° C., preferably at 5 to 45° C. and more preferably selected from 20 to 30° C.

The crystalline Raltegravir potassium using in step (i) is selected from crystalline form (s) of Raltegravir potassium known in prior art.

The suitable premixing agent of step (ii) can be any pharmaceutically acceptable excipient(s) discussed in the specification includes but not limited to diluents, lubricants, disintegrants, glidants, stabilizers & surface active agents or mixtures thereof.

Exemplary premixing agents or pharmaceutically acceptable excipients used in step (ii) include, but are not limited topolyvinylpyrrolidone (also called povidone), polyvinyl alcohol, polyethylene glycol, polyol (Mannitol), sodium starch glycolate, colloidal silicon dioxide(aerosil), hydroxypropyl methylcellulose, methyl cellulose, carboxymethyl cellulose, sodium carboxymethyl cellulose, hydroxyethylcellulose, polyvinyl acetate, cyclodextrins, gelatins, hypromellose phthalate, sugars, and combinations comprising one or more of the foregoing agents, Preferable premixing agents selected from povidone, mannitol, polyethylene glycol (PEG) and aerosol 200.

Removal of solvent in step (iii) is accomplished, for example, by substantially complete evaporation of the solvent, concentrating the solution or distillation of solvent, under inert atmosphere to obtain the stable amorphous Raltegravir potassium premix.

In another embodiment, the solvent is removed by evaporation. Evaporation can be achieved at sub-zero temperatures by lyophilisation or freeze-drying techniques. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Solvents can also be removed by spray-drying, in which a solution comprising Raltegravir potassium and a premixing agent is sprayed into the spray drier at the flow rate ranging from 10 to 300 ml/hr, specifically 40 to 200 ml/hr. The air inlet temperature to the spray drier used may range from about 30° C. to about 150° C., specifically from about 65° C. to about 110° C. and the outlet air temperature used may range from about 30° C. to about 90° C.

Another suitable method is vertical agitated thin-film drying (or evaporation). Agitated thin film evaporation technology involves separating the volatile component using indirect heat transfer coupled with mechanical agitation of the flowing film under controlled conditions. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

The Raltegravir potassium with the premixing agent obtained by process disclosed herein may be further dried, preferably spin dried, in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In another embodiment, the present invention provides a pharmaceutical composition comprising stable amorphous Raltegravir potassium premix with pharmaceutically acceptable excipients.

The premix can be formulated into various pharmaceutical compositions like powder, granules, capsules, tablets, pellets etc.

The pharmaceutical composition of the invention can be formed by various methods known in the art such as by dry granulation, wet granulation, melt granulation, direct compression, double compression, extrusion spheronization, layering and the like. The composition or formulation may be coated or uncoated. Coating of compositions such as tablets and caplets is well known in the art.

Pharmaceutically acceptable excipients may be utilized as required for conversion of the premixes into the final pharmaceutical dosage forms and include, for example, any one or more of diluents, binders, stabilizers, lubricants, glidants, disintegrating agents, surfactants, and other additives that are commonly used in solid pharmaceutical dosage form preparations.

### Diluents:

Various useful fillers or diluents include but are not limited to starches, lactose, mannitol (Pearlitol™ SD200), cellulose derivatives, confectioner's sugar and the like. Different grades of lactose include but are not limited to lactose monohydrate, lactose DT (direct tableting), lactose anhydrous, Flowlac™, Pharmatose™ and others. Different starches include but are not limited to maize starch, potato starch, rice starch, wheat starch, pregelatinized starch and starch 1500, starch 1500 LM grade (low moisture content grade) from Colorcon, fully pregelatinized starchand others. Different cellulose compounds that can be used include crystalline celluloses and powdered celluloses. Examples of crystalline cellulose products include but are not limited to CEOLUS™ KG801, Avicel™ PH101, PH102, PH301, PH302 and PH-F20, PH112 microcrystalline cellulose 114, and microcrystalline cellulose 112. Other useful diluents include but are not limited to carmellose, sugar alcohols such as mannitol (Pearlitol™ SD200), sorbitol and xylitol, calcium carbonate, magnesium carbonate, dibasic calcium phosphate, and tribasic calcium phosphate.

### Binders:

Various useful binders include but are not limited to hydroxypropyl celluloses, also called HPC (Klucel™ LF, Klucel EXF) and useful in various grades, hydroxypropylmethylcelluloses, also called hypromelloses or HPMC (Methocel™) and useful in various grades, polyvinylpyrrolidones or povidones (such as grades PVP-K25, PVP-K29, PVP-K30, and PVP-K90), Plasdone™ S-630 (copovidone), powdered acacia, gelatin, guar gum, carbomers (Carbopol™), methylcelluloses, polymethacrylates, and starches.

### Disintegrants:

Various useful disintegrants include but are not limited to carmellose calcium, carboxymethylstarch sodium, croscarmellose sodium, crospovidones, examples of commercially available crospovidone products including but not limited to crosslinkedpovidone, Kollidon™ CL, Polyplasdone™ XL, XI-10, and INF-10 and low-substituted hydroxypropylcelluloses. Examples of low-substituted hydroxypropylcelluloses include but are not limited to low-substituted hydroxypropylcellulose LH11, LH21, LH31, LH22, LH32, LH20, LH30, LH32 and LH33. Other useful disintegrants include sodium starch glycolate, colloidal silicon dioxide, and starches.

### Stabilizers:

Various useful stabilizers include basic inorganic salts, such as but not limited to basic inorganic salts of sodium, potassium, magnesium and calcium. Examples of basic inorganic salts of sodium are sodium carbonate, sodium hydrogen carbonate, sodium hydroxide, and the like. Examples of basic inorganic salts of potassium are potassium carbonate, potassium hydrogen carbonate, potassium hydroxide, and the like. Examples of basic inorganic salts of magnesium are heavy magnesium carbonate, magnesium carbonate, magnesium oxide, magnesium hydroxide, magnesium metasilicate aluminate, magnesium silicate, magnesium aluminate, synthetic hydrotalcite [Mg₆Al₂(OH)₁₆.CO₃.4H₂O], aluminum hydroxide-magnesium[2.5MgO.Al₂O₃.xH₂O], and the like. Examples of basic inorganic salts of calcium include precipitated calcium carbonate, calcium hydroxide, and the like.

### Surface-Active Agents:

Useful surface-active agents include non-ionic, cationic and anionic surface-active agents. Useful non-ionic surface-active agents include ethylene glycol stearates, propylene glycol stearates, diethylene glycol stearates, glycerol stearates, sorbitan esters (SPAN™) and polyhydroxyethylenically treated sorbitan esters (TWEEN™), aliphatic alcohols and PEG ethers, phenol and PEG ethers. Useful cationic surface-active agents include quaternary ammonium salts (e.g. cetyltrimethylammonium bromide) and amine salts (e.g. octadecylamine hydrochloride). Useful anionic surface-active agents include sodium stearate, potassium stearate, ammonium stearate, and calcium stearate, triethenolamine stearate, sodium lauryl sulphate, sodium dioctylsulphosuccinate, and sodium dodecylbenzenesulphonate. Natural surface-active agents may also be used, such as for example phospholipids, e.g. diacylphosphatidylglycerols, diaceylphosphatidylcholines, and diaceylphosphatidic acids, the precursors and derivatives thereof, such as for example soybean lecithin and egg yolk.

### Lubricants:

An effective amount of any pharmaceutically acceptable tableting lubricant can be added to assist with compressing tablets. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

### Glidants:

One or more glidant materials, which improve the flow of powder blends and minimize dosage form weight variations can be used. Useful glidants include but are not limited to silicone dioxide, talc and combinations thereof.

### Coloring Agents:

Coloring agents can be used to color code the compositions, for example, to indicate the type and dosage of the therapeutic agent therein. Suitable coloring agents include, without limitation, natural and/or artificial compounds such as FD&C coloring agents, natural juice concentrates, pigments such as titanium oxide, silicon dioxide, iron oxides, zinc oxide, combinations thereof, and the like.

Useful additives for coatings include but are not limited to plasticizers, antiadherents, opacifiers, solvents, and optionally colorants, lubricants, pigments, antifoam agents, and polishing agents.

Various useful plasticizers include but are not limited to substances such as castor oil, diacetylated monoglycerides, dibutylsebacate, diethyl phthalate, glycerin, polyethylene glycol, propylene glycol, triacetin, and triethyl citrate. Also, mixtures of plasticizers may be utilized. The type of plasticizer depends upon the type of coating agent. An opacifier like titanium dioxide may also be present, typically in an amount ranging from about 10% to about 20% based on the total weight of the coating.

The present invention includes administration of an effective amount of stable amorphous Raltegravir potassium premix (either alone or as the active component of a pharmaceutical composition) for inhibiting HIV integrase, for the treatment or prophylaxis of HIV infection, or for the treatment, prophylaxis, or delay in the onset of AIDS to a subject in need of such inhibition, treatment, prophylaxis, or delay.

The present invention also includes the use of a stable amorphous Raltegravir potassium premix in combination with an anti-HIV agent.

The invention is further illustrated by following examples, which should not be construed as limiting to the scope of invention.

### EXAMPLES

The X-ray diffraction patterns were measured using Philips X′Pertpro machine with following measurement parameters:

Scan axis: Gonio
Step size: 0.0080°
Scan type: continuous
DLlergence slit size: 0.2393°
Anode material: Cu
Radiation type: K-alpha 1

### Scan: 3.49 to 40° 2θ

### Spinning: Yes

Measurement temperature: 25° C.
Stability Data Comparison Raltegravir Potassium with Premix and without Premix:

| | | Initial | After 1 | After 2 | After 3 |
|---|---|---|---|---|---|
| | Storage | Purity | month | months | months |
| API | conditions | (%) | (%) | (%) | (%) |
| Raltegravir | Temp: 5 ± 3° C. | 99.89 | 98.66 | — | — |
| potassium | | | | | |
| (without | | | | | |
| premix) | | | | | |
| Raltegravir | Temp: 25 ± 2° C. | 99.54 | 99.6 | 99.7 | 99.4 |
| potassium with | Rh: 60 ± 5% | | | | |
| 10% PEG4000 | | | | | |
| Raltegravir | Temp: 25 ± 2° C. | 99.8 | 99.9 | 99.3 | 99.2 |
| potassium with | Rh: 60 ± 5% | | | | |
| 5% Mannitol | | | | | |

### Example 1

### Preparation of Amorphous Raltegravir Potassium Premix with Mannitol

20 g of crystalline Raltegravir potassium form-I was suspended in 300 ml of millipore water at 20° C. and stirred for 60 minutes to get clear solution. Next, premix agent, 1.0 g of mannitol was added. The resulting mass was further stirred for 10 minutes at 20 to 25° C., filtered the reaction mass and concentrated filtrate in rotavapor apparatus under vacuum at 45 to 55° C. to get solid. Then dried the obtained solid in rotavapor apparatus under vacuum at 60° C. to get 17.96 g (89.8%) of amorphous Raltegravir potassium premix with mannitol was obtained.

HPLC purity=99.8%

### Example 2

### Preparation of Amorphous Raltegravir Potassium Premix with PEG-4000

20 g of crystalline Raltegravir potassium form-I was suspended in 300 ml of millipore water at 20° C. and stirred for 10 minutes to get clear solution. Next, premix agent, 2.0 g of PEG-4000 was added. The resulting mass was further stirred for 60 minutes at 20 to 25° C., filtered the reaction mass and concentrated filtrate in rotavapor apparatus under vacuum at 45 to 55° C. to get solid. Then dried the obtained solid in rotavapor apparatus under vacuum at 60° C. to get 19.75 g (98.75%) of amorphous Raltegravir potassium premix with PEG-4000 was obtained.

HPLC purity=99.54%

### Example 3

### Preparation of Amorphous Raltegravir Potassium Premix with Aerosil 200

20 g of crystalline Raltegravir potassium form-I was suspended in 300 ml of millipore water at 20° C. and stirred for 10 minutes to get clear solution. Next, premix agent, 1.0 g of Aerosil 200 was added. The resulting mass was further stirred for 60 minutes at 20 to 25° C., filtered the reaction mass and concentrated filtrate in rotavapor apparatus under vacuum at 45 to 55° C. to get solid. Then dried the obtained solid in rotavapor apparatus under vacuum at 60° C. to get 17.55 g (87.75%) of amorphous Raltegravir potassium premix with PEG-4000 was obtained.

HPLC purity=99.66%

1. A stable amorphous Raltegravir potassium premix comprising Raltegravir potassium and at least one pharmaceutically acceptable excipient.

2. The stable amorphous Raltegravir potassium premix of claim 1, wherein the pharmaceutically acceptable excipient can be a diluent, lubricant, disintegrant, stabilizer, glidant or surface active agent or mixtures thereof.

3. The stable amorphous Raltegravir potassium premix of claim 2, wherein a pharmaceutically acceptable excipient is selected from the group consisting of polyvinylpyrrolidone (also called povidone), polyvinyl alcohol, polyethylene glycol, polyol (Mannitol), sodium starch glycolate, colloidal silicon dioxide(aerosil), hydroxypropyl methylcellulose, methyl cellulose, carboxymethyl cellulose, sodium carboxymethyl cellulose, hydroxyethylcellulose, polyvinyl acetate, cyclodextrins, gelatins, hypromellose phthalate, sugars and mixtures thereof.

4. A process for preparing stable amorphous Raltegravir potassium premix comprising the steps of:
(i) providing a solution of crystalline Raltegravir potassium in a solvent;
(ii) adding suitable premixing agent(s); and
(iii) substantially removing the solvents from the solution to afford stable amorphous Raltegravir potassium premix.

5. The process of claim 4, wherein a solvent of step i) is selected from the group consisting of dichloromethane, 1,2-dichloroethane, chloroform, carbon tetrachloride, methanol, ethanol, isopropyl alcohol, 1-propanol, 2-propanol, 1-butanol, 2-butanol, and t-butyl alcohol, acetone, ethyl methyl ketone, diethyl ketone, and methyl isobutyl ketone, ethyl acetate, n-propyl acetate, n-butyl acetate and t-butyl acetate, diethyl ether, dimethyl ether, diisopropyl ether, methyl t-butyl ether and 1,4-dioxane, acetonitrile, propionitrile, water and mixtures thereof.

6. The process of claim 4, wherein the premixing agent of step ii) is selected from the group consisting of polyvinylpyrrolidone (also called povidone), polyvinyl alcohol, polyethylene glycol, polyol (Mannitol), sodium starch glycolate, colloidal silicon dioxide(aerosil), hydroxypropyl methylcellulose, methyl cellulose, carboxymethylcellulose, sodium carboxymethyl cellulose, hydroxyethylcellulose, polyvinyl acetate, cyclodextrins, gelatins, hypromellose phthalate, sugars and mixtures thereof.

7. The process of claim 4, wherein the removal of the solvent in step iii) is accomplished by complete evaporation of the solvent, concentrating the solution or distillation, spray drying, vacuum drying, lyophilization or freeze drying, agitated thin-film (ATFD) drying, or a combination thereof.

8. A pharmaceutical formulation comprising stable amorphous Raltegravir potassium premix of claim 1 and at least one pharmaceutically acceptable excipient.

9. The pharmaceutical formulation of claim 8, is in the form of a tablet, capsule, powder, caplet, granules, pellets, tablet in tablet, tablet in capsule, pellets in capsule, powder in capsule and granules in capsule.

10. A method for treating or inhibiting HIV integrase, prophylaxis of HIV infection comprising administering a pharmaceutical composition that comprises a therapeutically effective amount of the stable amorphous Raltegravir potassium premix of claim 1, along with additional pharmaceutically acceptable excipient and optionally combine with other anti-HIV agent.
